(19) United States
(12) Patent Application Publication
(10) Pub. No.: US 2025/0302439 A1
(43) Pub. Date: Oct. 2, 2025

(54) HYBRID INTRAVASCULAR ULTRASOUND AND INTRACARDIAC ECHOCARDIOGRAPHY TRANSDUCER AND ASSOCIATED DEVICES, SYSTEMS, AND METHODS

(71) Applicant: KONINKLIJKE PHILIPS N.V., EINDHOVEN (NL)

(72) Inventor: THOMAS JOHN MCPEAK, CALEDONIA, MN (US)

(21) Appl. No.: 18/872,868

(22) PCT Filed: Jun. 12, 2023
(86) PCT No.: PCT/EP2023/065567

§ 371 Date: Dec. 9, 2024

Related U.S. Application Data
(60) Provisional application No. 63/354,879, filed on Jun. 23, 2022.

(51) Int. Cl. A61B 8/00; A61B 8/08; A61B 8/12; B06B 1/02; F03G 7/06

(52) U.S. Cl. CPC A61B 8/445; A61B 8/0883; A61B 8/12; A61B 8/4488; A61B 8/4494; B06B 1/0292; F03G 7/0614; B06B 2201/76

(57) ABSTRACT

An intraluminal imaging device includes a flexible elongate member that can be positioned within a patient. The device includes an ultrasound imaging assembly coupled to a distal portion of the flexible elongate member. The ultrasound imaging assembly includes an expandable support member with a first state and a second state. The ultrasound imaging assembly includes a transducer array coupled to the expandable support member. In the first state of the expandable support member, the transducer array has a first shape. such as a substantially cylindrical shape suitable for intravascular ultrasound (IVUS) imaging within a blood vessel. In the second state of the expandable support member. the transducer array has a second shape. such as a substantially planar or hemicylindrical shape suitable for intracardiac echography (ICE) imaging within a heart chamber.

### TECHNICAL FIELD

The present disclosure relates generally to intraluminal ultrasound imaging and, in particular, to the structure of an ultrasound imaging assembly at a distal portion of a catheter or guidewire. For example, an ultrasound imaging assembly is transitionable from a cylindrical configuration for intravascular imaging to a convex or flat configuration for intracardiac imaging.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess a treatment's effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed. IVUS imaging is can be performed using a one-dimensional (1D) ultrasound imaging array that has been rolled and fixed in a cylindrical shape.

Intracardiac echography (ICE) imaging is used to obtain images of the interior of the heart. An ICE catheter also relies on transducers that emit ultrasound energy and receive ultrasound echoes. However, where IVUS relies on a cylindrical, one-dimensional (1D) transducer array to capture two-dimensional (2D) images, ICE can use a 2D transducer array to capture images. Some procedures can require both IVUS and ICE imaging, which may necessitate removal of an IVUS catheter from the body and replacement with an ICE catheter.

### SUMMARY

Disclosed herein is an intraluminal catheter with a hybrid or transitionable ultrasound transducer array that can perform both IVUS imaging when positioned inside a blood vessel and ICE imaging when positioned inside of a heart chamber. The ultrasound transducer array is built on a flexible substrate, mounted on a deployable and retrievable structure, which may for example be similar to a retrievable stent, such that it is cylindrical when in IVUS mode, and unfurls to a flat or convex shape in ICE mode.

Because some medical procedures may require the use of both IVUS and ICE imaging, the ability to provide both IVUS and ICE imaging capabilities with a single imaging catheter may enable savings not only of cost, but also the time required to remove an IVUS catheter and insert an ICE catheter (or vice versa). This may reduce the time required to perform some procedures, and thus the time required for the patient to be under anesthesia and/or x-ray.

In an exemplary aspect, an intraluminal imaging device is provided. The device includes a flexible elongate member configured to be positioned within a patient; and an ultrasound imaging assembly coupled to a distal portion of the flexible elongate member and comprising: an expandable support member comprising a first state and a second state; and a transducer array coupled to the expandable support member, wherein, in the first state of the expandable support member, the transducer array comprises a first shape, and in the second state of the expandable support member, the transducer array comprises a second shape.

In some aspects, the expandable support member comprises a shape memory alloy. In some aspects, the flexible elongate member comprises a tension wire, and changing a tension of the tension wire causes the expandable support member to change from the first state to the second state. In some aspects, the transducer array comprises a first edge and an opposite second edge. In some aspects, the first shape is substantially cylindrical such that the first edge is in contact with the second edge or proximate to and facing substantially toward the second edge. In some aspects, the second shape is a planar or open arcuate shape such that the first edge is spaced from, and not facing toward, the second edge. In some aspects, the transducer array comprises a plurality of capacitive micromachined ultrasonic transducers (CMUTs). In some aspects, the first shape of the transducer array comprises an intravascular ultrasound (IVUS) configuration. In some aspects, the second shape of the transducer array comprises an intracardiac echography (ICE) configuration. In some aspects, the transducer array is a two-dimensional (2D) transducer array. In some aspects, the imaging assembly further comprises a flexible substrate coupled to the expandable support member, and the transducer array coupled to the flexible substrate. In some aspects, the flexible elongate member further comprises a pullwire coupled to and configured to deflect the distal portion of the flexible elongate member. In some aspects, a diameter of the transducer array is larger in the second shape of the transducer array than in the first shape of the transducer array. In some aspects, the expandable support member comprises a first plurality of arms that are attached to the transducer array and a second plurality of arms that are not attached to the transducer array.

In an exemplary aspect, a method is provided. The method includes providing an intraluminal imaging device comprising: a flexible elongate member configured to be positioned within a first body lumen and a second body lumen of a patient; and an ultrasound imaging assembly coupled to a distal portion of the flexible elongate member and comprising: an expandable support member in a first state; and a transducer array coupled to the expandable support member such that, in the first state of the expandable support member, the transducer array comprises a first shape for imaging while positioned within the first body lumen; and transitioning the expandable support member to second state such that the transducer array comprises a second shape for imaging while positioned within the second body lumen.

In an exemplary aspect, an ultrasound imaging device, comprising: a flexible elongate member configured to be positioned within a blood vessel and a heart chamber of a patient; and an ultrasound imaging assembly coupled to a distal portion of the flexible elongate member and comprising: an expandable support member; a tension wire configured to change the expandable support member between a first state of expansion and a second state of expansion; and a transducer array coupled to the expandable support member; wherein, in the first state of expansion of the expandable support member, the transducer array is in a substantially cylindrical shape suitable for intravascular ultrasound (IVUS) imaging within the blood vessel, and in the second state of expansion of the expandable support member, the transducer array is in a substantially planar or hemicylindrical shape suitable for intracardiac echography (ICE) imaging within the heart chamber.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the hybrid ultrasound device, as defined in the claims, is provided in the following written description of various embodiments of the disclosure and illustrated in the accompanying drawings.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2A is a cross-sectional side view of a human heart according to aspects of the present disclosure.

FIG. 2B is a cross-sectional side view of a human heart according to aspects of the present disclosure.

FIG. 3A is an IVUS image of a blood vessel that could, for example, represent the viewing region of the intraluminal imaging device in its IVUS configuration according to aspects of the present disclosure.

FIG. 3B is an ICE image of a heart valve that could, for example, represent the viewing region of the intraluminal imaging device in its ICE configuration (see FIG. 2B), according to aspects of the present disclosure.

FIG. 4 is a schematic, diagrammatic side view of at least a portion of an example transitionable intraluminal imaging device, according to aspects of the present disclosure.

FIG. 5 is a schematic, diagrammatic top view of an example imaging array in a flattened configuration, according to aspects of the present disclosure.

FIG. 6 is a schematic, diagrammatic, front cross-sectional view of an example imaging array in the compressed, unexpanded, or IVUS configuration, according to aspects of the present disclosure.

FIG. 7 is a diagrammatic, perspective side view of at least a portion of an example imaging array and expandable support member in the expanded or ICE configuration, according to aspects of the present disclosure.

FIG. 8 is a diagrammatic, front cross-sectional view of at least a portion of an example imaging array and expandable support member in the expanded or ICE configuration, according to aspects of the present disclosure.

FIG. 9 is a diagrammatic, front cross-sectional view of at least a portion of an example imaging array and expandable support member in the expanded or ICE configuration, according to aspects of the present disclosure.

FIG. 10 is a diagrammatic, side cross-sectional view of an example imaging array in the compressed, unexpanded, or IVUS configuration, according to aspects of the present disclosure.

FIG. 11 is a diagrammatic, side cross-sectional view of at least a portion of an example imaging array and expandable support member in an expanded or ICE configuration, according to aspects of the present disclosure.

FIG. 12 is a diagrammatic, side cross-sectional view of at least a portion of an example imaging array and expandable support member in an expanded or ICE configuration, according to aspects of the present disclosure.

FIG. 13 is a side perspective view of at least a portion of an example hybrid intraluminal imaging device in an expanded or ICE configuration, according to aspects of the present disclosure.

FIG. 14 is a diagrammatic, side perspective view of the viewing region of an example hybrid intraluminal imaging device in the compressed, unexpanded, cylindrical, or IVUS configuration, according to aspects of the present disclosure.

FIG. 15 is a diagrammatic, side perspective view of the viewing region of an example hybrid intraluminal imaging device in the expanded or ICE configuration, according to aspects of the present disclosure.

FIG. 16 is a longitudinal cross-sectional view of at least a portion of an example body lumen, according to aspects of the present disclosure.

FIG. 17 is a flow diagram of an example hybrid ultrasound imaging method,

according to aspects of the present disclosure.

FIG. 18 is a schematic diagram of a processor circuit, according to aspects of the present disclosure.

### DETAILED DESCRIPTION

Disclosed herein is a catheter with a hybrid or transitionable ultrasound transducer array that can function as both an IVUS and an ICE imaging system. The ultrasound transducer array is built on a flexible substrate, mounted on a deployable and retrievable structure, which may for example be similar to a retrievable stent, such that it is cylindrical when in IVUS mode, and unfurls to a flat or convex shape in ICE mode.

In IVUS mode, the hybrid or transitionable transducer array can for example be used to image the vasculature from the access point to the heart, as well as sizing the annular structures of valves. When unfurled in ICE mode, it can for example be used to look at valves enface, as well as looking for regurgitation or leaks using doppler ultrasound.

The hybrid imaging catheter could be used for planning purposes, diagnostically, or for confirmation post procedure.

Furthermore, the transducer array may be constructed using CMUT or similar technology, in order to optimize the frequency of operation in either mode. For example, one might want to lower the frequency in IVUS mode in order to gain depth penetration, or increase the frequency while in ICE mode to get more resolution. Currently, catheters only function as an IVUS or an ICE catheter. The ability to make an ultrasound transducer that is flexible or conformable has been demonstrated. The present disclosure takes advantage of the flexibility of the transducer to be able to function in different “modes”, allowing for the optimization of the imaging for different purposes.

This may for example reduce or eliminate the need to have two different catheters for different purposes in the same procedure. It is expected that the cost of this hybrid catheter would be less than the cost of an IVUS and an ICE catheter obtained separately. The user interface for the ICE mode would also be expected to be simplified over the user interface for an ICE catheter, both from a catheter manipulation and a software interface perspective.

A flexible ultrasound transducer mounted on a retrievable structure that allows for the transducer to be cylindrical in one instance, and a more flat, open, rectangular shape in the other. It may be desirable to be able to retrieve the rectangular shaped sensor back into the cylindrical shape in order to remove the catheter from the patient.

A catheter equipped with the hybrid or transitionable transducer array may for example be deflectable in at least one direction, and may have a deployable/retrievable element similar to a retrievable stent or thrombosis retrieval device, with an ultrasound transducer mounted on it.

In the case where the transducer array is cylindrical, the catheter can function as a normal IVUS catheter, and may be used to image vessels, measure the diameter of the annulus of a heart valve, etc. In the instance where the transducer array is unfurled, it can function as an ICE catheter, and allow the operator to image valves enface, and measure/detect leaks and regurgitation using doppler ultrasound. However, the user interface could be much less complicated both from a catheter interface and software perspective.

This technology is applicable to the process of diagnosing, treating, and confirming the successful treatment of structural heart diseases and could also be applied to peripheral vascular disease as well. Since some medical procedures may require the use of both IVUS and ICE imaging, the ability to provide both IVUS and ICE imaging capabilities with a single imaging catheter may enable savings not only of cost, but also the time required to remove an IVUS catheter and insert an ICE catheter. This may reduce the time required to perform some procedures, and thus the time required for the patient to be under anesthesia.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

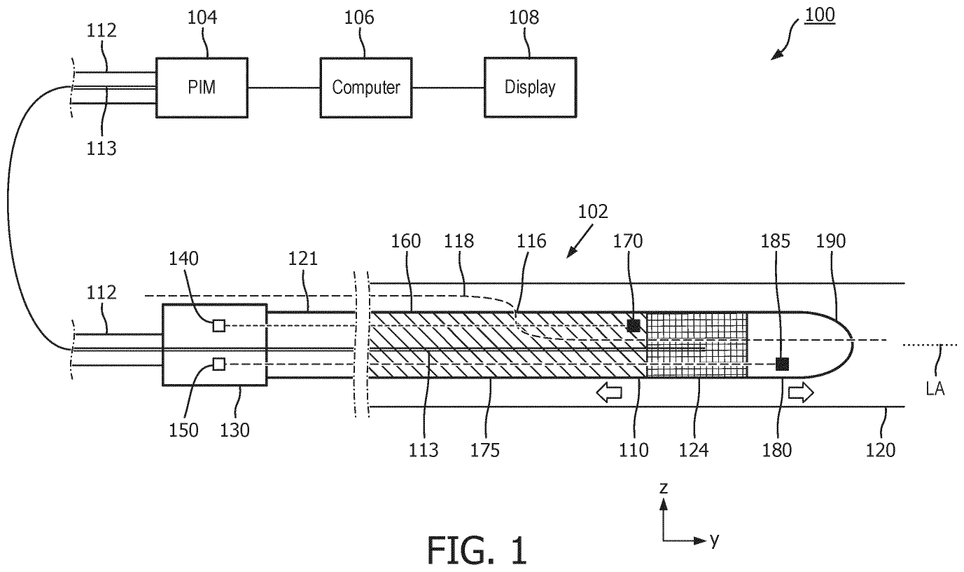

FIG. 1 is a diagrammatic schematic view of an ultrasound imaging system 100, according to aspects of the present disclosure. The ultrasound imaging system 100 can be an intraluminal imaging system. In some instances, the system 100 can be configured as an intravascular ultrasound (IVUS) imaging system, while in other instances the same system 100 can be reconfigured as an intracardiac echography (ICE) imaging system. The system 100 may include an intraluminal imaging device 102 such as a catheter, guide wire, or guide catheter, a patient interface module (PIM) 104, a processing system or console 106, and a monitor 108. The intraluminal imaging device 102 can be an ultrasound imaging device. In some instances, the device 102 can be configured as an IVUS imaging device, while in other instances, the same intraluminal imaging device 102 can be reconfigured as an ICE imaging device.

At a high level, the imaging device 102 emits ultrasonic energy, or ultrasound signals, from a transducer array 124 included in scanner assembly or scanner body 110 mounted near a distal end of the catheter device. The ultrasonic energy is reflected by tissue structures in the medium, such as a blood vessel or other body lumen 120 surrounding the scanner assembly or scanner body 110, and the ultrasound echo signals are received by the transducer array 124. In that regard, the intraluminal imaging device 102 can be sized, shaped, or otherwise configured to be positioned within the body lumen of a patient. The PIM 104 transfers the received echo signals to the console or computer 106, where the ultrasound image is reconstructed and displayed on the monitor 108. The console or computer 106 can include a processor and a memory. The computer or processing system 106 can be operable to facilitate the features of the ultrasound imaging system 100 described herein. For example, the processor can execute computer readable instructions stored on the non-transitory tangible computer readable medium.

The PIM 104 facilitates communication of signals between the computer or console 106 and the scanner assembly 110 included in the imaging device 102. This communication includes the steps of: (1) providing commands to integrated circuit controller chip(s) included in the scanner assembly 110 to select the particular transducer array element(s), or acoustic element(s), to be used for transmit and receive, (2) providing the transmit trigger signals to the integrated circuit controller chip(s) included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) of the scanner assembly 110. In some embodiments, the PIM 104 performs preliminary processing of the echo data prior to relaying the data to the computer, console, or processing system 106. In examples of such embodiments, the PIM 104 performs amplification, filtering, and/or aggregating of the data. In an embodiment, the PIM 104 also supplies high-and low-voltage DC power to support operation of the device 102 including circuitry within the scanner assembly 110.

The ultrasound console 106 receives the echo data from the scanner assembly 110 by way of the PIM 104 and processes the data to reconstruct an image of the tissue structures in the medium surrounding the scanner assembly 110. Generally, the device 102 can be utilized within any suitable anatomy and/or body lumen of the patient. The computer, console, or processing system 106 outputs image data such that an image of the vessel or lumen 120, such as a cross-sectional IVUS image of the lumen 120 or a three-dimensional (e.g., pyramidal) ICE image of the body lumen 120, is displayed on the monitor 108. Lumen 120 may represent fluid filled or surrounded structures, both natural and man-made. Lumen 120 may be within a body of a patient. Lumen 120 may be a blood vessel, such as an artery or a vein of a patient's vascular system, including cardiac vasculature, peripheral vasculature, neural vasculature, renal vasculature, and/or or any other suitable lumen inside the body. Body lumen 120 may also be a chamber or the heart, or another orgian of the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the imaging device 102 includes some features similar to traditional solid-state IVUS catheters, such as the EagleEye® catheter available from Volcano Corporation and those disclosed in U.S. Pat. No. 7,846,101 hereby incorporated by reference in its entirety. For example, the imaging device 102 includes the scanner assembly 110 near a distal end of the device 102 and a transmission line bundle 112 extending along the longitudinal body of the device 102 within a flexible elongate member 121. It is understood that any suitable gauge wire can be used for the transmission line bundle 112. In an embodiment, the transmission line bundle 112 can include a four-conductor transmission line arrangement with, e.g., 41 American wire gauge (AWG) gauge wires. In an embodiment, the cable or transmission line bundle 112 can include a seven-conductor transmission line arrangement utilizing, e.g., 44 AWG gauge wires. In some embodiments, 43 AWG gauge wires can be used. Thus, the electrical cable or transmission line bundle 112 can contain a plurality of electrical wires or conductors 113.

The transmission line bundle is physically and electrically coupled to the PIM 104 (e.g., with a connector). In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

An ultrasound transducer array of ultrasound imaging device includes an array of acoustic elements configured to emit ultrasound energy and receive echoes corresponding to the emitted ultrasound energy. In some instances, the array may include any number of ultrasound transducer elements. For example, the array can include between 2 acoustic elements and 10000 acoustic elements, including values such as 2 acoustic elements, 4 acoustic elements, acoustic elements, 64 acoustic elements, 128 acoustic elements, 500 acoustic elements, 812 acoustic elements, 3000 acoustic elements, 9000 acoustic elements, and/or other values both larger and smaller. In some instances, the transducer elements of the array may be arranged in any suitable configuration, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

The ultrasound transducer elements may comprise piezoelectric/piezoresistive elements (e.g., PZT), piezoelectric micromachined ultrasound transducer (PMUT) elements, capacitive micromachined ultrasound transducer (CMUT) elements, and/or any other suitable type of ultrasound transducer elements. The ultrasound transducer elements of the array are in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can comprise a microbeamformer (μBF). In other embodiments, one or more of the ICs comprises a multiplexer circuit (MUX).

The intraluminal imaging device 102 includes a handle 130, which includes actuators for manipulating the intraluminal imaging device 102. In the example shown in FIG. 1, the handle 130 includes a deflection actuator 140 and a deployment actuator 150. Depending on the implementation, the actuators 140, 150 may comprise dials, switches, levers, knobs, motors, or otherwise.

The deflection actuator 140 is coupled to a pull wire 160 that is anchored to an anchor point 170 on a side of the flexible elongate member 121, such that when the pull wire 160 is pulled by the deflection actuator 140, the anchor point 170 moves with the pull wire 160, and an articulation section 175 of the flexible elongate member 121 is bent or deflected away from a longitudinal axis LA. Similarly, when the pull wire 160 is relaxed by the deflection actuator 140, the flexible elongate member 121 returns to a straight configuration (e.g., aligned with the longitudinal axis LA, or with the body lumen, or otherwise). In some embodiments, multiple deflection actuators 140, pull wires 160, and anchor points 170 may be provided, to bend the flexible elongate member 121 in a plurality of different directions. This may for example help navigate the imaging device 102 through tortuous vasculature, or may help align the imaging array 124 with an anatomical region of interest.

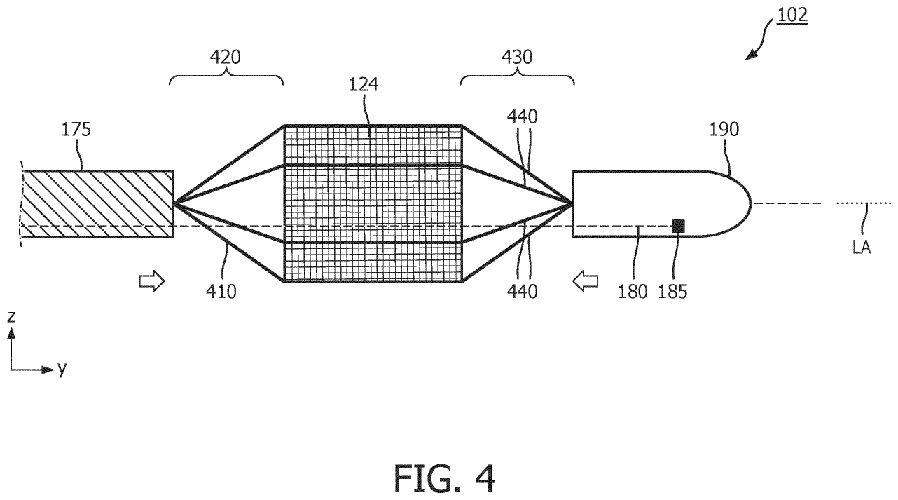

The deployment actuator 150 is coupled to a tension wire 180 that anchors to an anchor point 185 at a distal tip 190 of the intraluminal imaging device 102. An expanding mechanism coupled to the ultrasound imaging array 124 (see FIG. 4) is held in a compressed state by tension on the tension wire 180, but can naturally expand into an expanded state if tension is loosened on the tension wire 180. Thus, the deployment actuator 150 is capable of transitioning the ultrasound imaging array 124 from a closed, cylindrical IVUS configuration (e.g., a first state of expansion) when the tension wire 180 is tight, to an open, flat or arc-shaped ICE configuration (e.g., a second state of expansion) when the tension wire 180 is loosened. Since some medical procedures may require the use of both IVUS and ICE imaging, the ability to provide both IVUS and ICE imaging capabilities with a single imaging catheter may enable savings not only of cost, but also the time required to remove an IVUS catheter and insert an ICE catheter. This may reduce the time required to perform some procedures, and thus the time required for the patient to be under anesthesia.

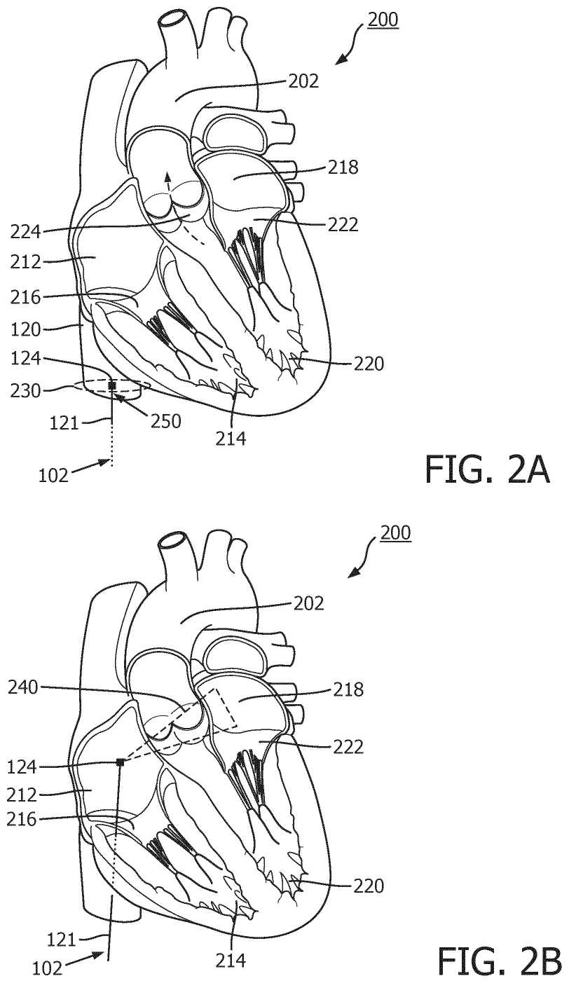

FIG. 2A is a cross-sectional side view of a human heart 200 according to aspects of the present disclosure. Visible are a right atrium 212 and a right ventricle 214. In that regard, oxygen-poor blood enters the human heart 200 in the right atrium 212 and travels to the right ventricle 214 through the tricuspid valve 216. The oxygen-poor blood leaves the right ventricle 214 and travels to the lungs. Also visible are a left atrium 218 and a left ventricle 220. In that regard, oxygen-rich blood is received from the lungs in the left atrium 218 and travels to the left ventricle 220 through the mitral valve 222. The oxygen-rich blood leaves the left ventricle 220 and goes out to the body through the aorta 202 via an aortic valve 224.

In the example shown in FIG. 2A, an intraluminal imaging device 102 has been inserted into a blood vessel 120 proximate to the heart 200. At a distal portion 250 of the flexible elongate member 121, the ultrasound imaging array 124 is in an (e.g., closed cylindrical) IVUS configuration, with a viewing region 230 that is a flat (e.g., two-dimensional) disc orthogonal to the longitudinal axis of the flexible elongate member 121, e.g., for imaging of the blood vessel 120. It is understood that in the IVUS configuration, the intraluminal imaging device 102 can be used to image other body lumens than those shown here, including portions of the heart or other organs.

FIG. 2B is a cross-sectional side view of a human heart 200 according to aspects of the present disclosure. Visible are a right atrium 212, right ventricle 214, tricuspid valve 216, left atrium 218, left ventricle 220, mitral valve 222, aorta 202, and aortic valve 224.

In the example shown in FIG. 2A, an intraluminal imaging device 102 has been inserted into chamber (e.g., the right atrium 212) of the heart 200. At the distal portion 250 of the flexible elongate member 121, the ultrasound imaging array 124 is in an (e.g., unfurled or partially unfurled) ICE configuration, with a viewing region 240 that is a three-dimensional (e.g., pyramidal or cone-shaped) region extending approximately orthogonally from the longitudinal axis of the flexible elongate member 121, e.g., for imaging of the aortic valve 224. It is understood that the intraluminal imaging device 102 can be used to image other portions of the heart 200, or other organs of the body.

One advantage of the transitionable or hybrid imaging device of the present disclosure is that that user can continuously and directly go from the position of the catheter in FIG. 2A for IVUS imaging to the position of the catheter in FIG. 2B for ICE imaging, and/or vice versa, depending on clinical need during an imaging procedure.

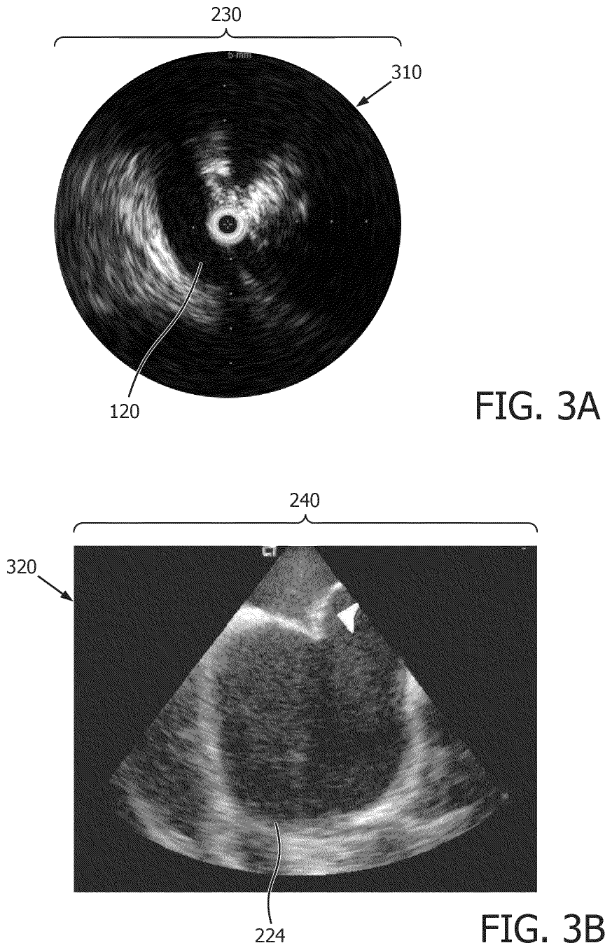

FIG. 3A is an IVUS image 310 of a blood vessel 120 that could, for example, represent the viewing region 230 of the intraluminal imaging device 102 in its IVUS configuration (see FIG. 2A according to aspects of the present disclosure. The IVUS image 310 is a 2D cross-section in a plane roughly perpendicular to the blood vessel 120, and can for example be captured by an imaging array that is rolled into a cylindrical shape.

FIG. 3B is an ICE image 320 of a heart valve 224 that could, for example, represent the viewing region 240 of the intraluminal imaging device 102 in its ICE configuration (see FIG. 2B), according to aspects of the present disclosure. The ICE image 320 is a 2D cross-section of a 3D (e.g., cone-shaped or pyramidal) imaging volume, and can for example be captured by an imaging array that is unfurled into a an open shape such as a plane, arc, or hemicylinder. 3D ICE images can also be generated from the 3D imaging volume.

FIG. 4 is a schematic, diagrammatic side view of at least a portion of an example transitionable intraluminal imaging device 102, according to aspects of the present disclosure. Visible are a portion of the articulation section 175 and distal tip 190 of the flexible elongate member, along with the tension wire 180 and its anchor point 185. The ultrasound imaging array 124 is mechanically coupled to an expandable support member 410 comprising a number of struts or arms 440 that may for example be coupled together at some locations (e.g., at the proximal and distal ends of the expandable support member 410), and may be free to move relative to one another at other locations to effectuate the expansion. The expandable support member 410 may for example be a stent-like structure made from a memory alloy material such as nitinol, and may be configured such that at room temperature it is normally in a first or compressed or unexpanded state, such that (for example) the articulation section 175 and the distal tip 190 are both proximate to the imaging array 124. However, at body temperature (e.g., inside a blood vessel or other body lumen) the expandable support member 410 may exceed a transition temperature such that it is normally in a second state or expanded state.

In the expanded state, the imaging array 124 is pushed away from the articulation section 175 along the longitudinal axis LA, such that a first gap 420 opens between the imaging array 124 and the articulation section 175. Similarly, in the expanded state the distal tip 190 is pushed away from the imaging array 124 along the longitudinal axis LA, such that a second gap 430 opens between the imaging array 124 and the distal tip 190. In an example, when the imaging array 124 is in the expanded state, it can be returned to the compressed or unexpanded state by applying tension to the tension wire 180, such that the distal tip 190 is pulled toward the articulation section 175 until both the distal tip 190 and the articulation section 175 are in contact with, or closely proximate to, the imaging array 124.

The imaging array 124 is coupled to the expandable support member 410, such that when the expandable support member 410 is in the compressed or unexpanded state, the imaging array 124 is wrapped entirely around the expandable support member 410 in a cylindrical shape (the IVUS configuration), whereas when the expandable support member 410 is in the expanded state, the imaging array 124 is not wrapped around the expandable support member 410, or is wrapped around only a portion of it, thus forming a plane, arc, or hemicylinder (the ICE configuration).

Depending on the implementation, the expandable support member 410 may be or may include other types of expanding structures, including but not limited to balloons, bladders, springs, etc., and may include different actuating mechanisms than the tension wire 180 and anchor point 185, including but not limited to pumps, hoses, motors, push rods, etc. In some embodiments, the expandable support member 410 may include a tubular section, split partially lengthwise (so that some tubular section remains on each end), forming it to splay out the split section, and setting it to that shape. Compression or expansion by the tip and shaft of the catheter could then force this splayed section to go back to the round, tubular shape. The tubular section could for example be plastic or metal (such as nitinol). Such a design may alleviate the need to laser cut a tube and then expand it to form an expandable stent-like structure.

In the expanded state, the imaging device 102 includes some features similar to ICE catheters, such as those described in U.S. Publication No. 2021/0298718, U.S. Publication No. 2022/0071590, U.S. Publication No. 2019/0307420, U.S. Publication No. 2021/0275136, U.S. Pat. No. 8,840,560, and U.S. Pat. No. 7,641,480, which are hereby incorporated by reference in their entireties.

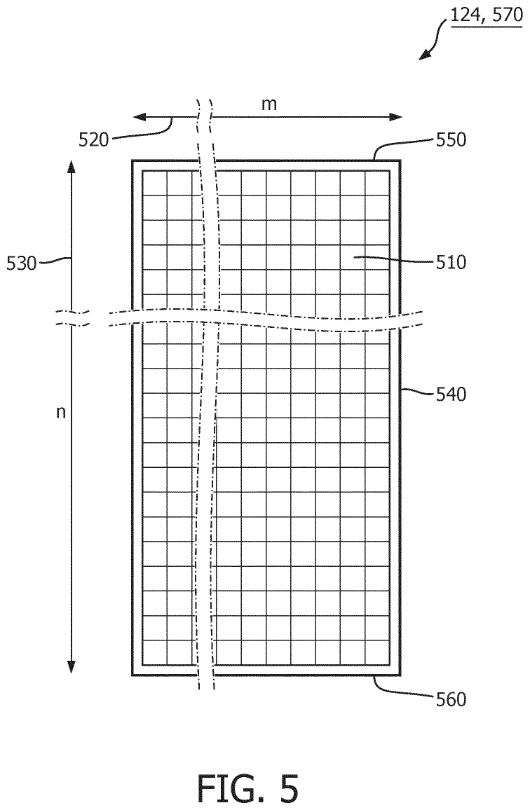

FIG. 5 is a schematic, diagrammatic top view of an example imaging array 124 in a flattened configuration, according to aspects of the present disclosure. In the example shown in FIG. 5, the imaging array 124 is a two-dimensional (2D) array of “m” transducer elements 510 along a first direction 520 and “n” transducer elements 510 along a second direction 530, for a total of m×n transducer elements 510. It is understood that IVUS imaging can be performed with a one-dimensional (1D) transducer array, such as a 1×n array, whereas ICE imaging may be performed using a 2D array that can produce three-dimensional (3D) images. In some cases, one row of a 2D array can be used as a 1D array. In other cases, IVUS imaging may be performed using a 2D array, such as a 2×n array, 3×n array, or larger. Thus, the m×n array shown in FIG. 5 may be usable for both IVUS and ICE imaging, depending on whether or not the imaging array 124 is rolled into a cylindrical shape.

In an example, the transducer elements 510 are mounted on a flexible substrate 540 such that the imaging array 124 is flexible on at least one axis, and preferably bendable or rollable around at least two orthogonal axes (e.g., directions 520 and 530). The imaging array 124 includes a first edge 550 and an opposite second edge 560. When the first direction 520 is parallel with the longitudinal axis of the flexible elongate member, the imaging array 124 can be rolled into a cylinder wherein the first edge 550 is parallel to and in contact with, or parallel to and closely proximate with, the second edge 560, thus forming the imaging array into the IVUS configuration. When the imaging array 124 is not fully cylindrical (e.g., when it is flat, as shown in FIG. 5, or when it is arc-shaped or hemicylindrical), it can be usable for ICE imaging, and can thus be considered to be in the ICE configuration 570.

Depending on the implementation, the transducer elements 510 may be piezoelectric micromachined ultrasound transducers (PMUT), capacitive micromachined ultrasound transducers CMUT, or other types of ultrasound transducers, or combinations thereof. In some embodiments, the transducer elements 510 may be photoacoustic/optoacoustic transducers, or other types of imaging transducers, without limitation. Where the transducer array is constructed using CMUT or similar technology, it can be used to optimize the frequency of operation in either IVUS or ICE mode. For example, one might want to lower the frequency in IVUS mode in order to gain depth penetration, or increase the frequency while in ICE mode to get more resolution (and/or vice versa).

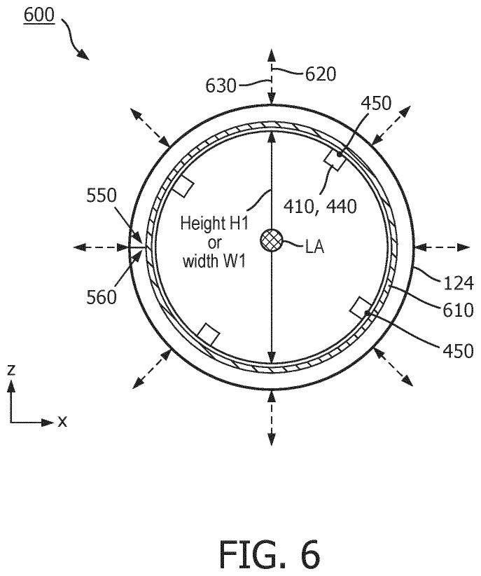

FIG. 6 is a schematic, diagrammatic, front cross-sectional view of an example imaging array 124 in the compressed, unexpanded, or IVUS configuration 600, according to aspects of the present disclosure. In the example shown in FIG. 6, the imaging array 124 is backed by an acoustic backing material 610, which may for example be attached to, or be part of, the flexible substrate 540 of the imaging array 124 (see FIG. 5). The imaging array 124 is supported by the struts or arms 440 of the expandable support member 410, and is attached to at least some of the arms 440 by attachment points 450, while some other arms 440 may not be attached to the imaging array 124. Depending on the implementation, the number and positions of attached vs. unattached arms may facilitate expansion of the expandable support member 410 without stretching of the imaging array 124. In the IVUS configuration 600, the imaging array 124 sends ultrasound energy 620 in a radially outward direction, and receives ultrasound echoes 630 in a radially inward direction.

In the compressed, unexpanded, or IVUS configuration 600, the imaging array 124 is rolled into a cylindrical shape that is at least approximately concentric with the longitudinal axis LA, such that the first edge 550 and the second edge 560 meet. The resulting cylindrical shape has a height H1 or width W1. In some cases, the first edge 550 and second edge 560 will be in linear contact across the entire length of the edges 550 and 560. In other cases, only portions of the edges 550 and 560 will be in contact. In still other cases, the first edge 550 and second edge 560 may be proximate to one another, and facing at least approximately toward one another, without actually physically contacting one another. Each of these configurations can be considered an embodiment of the IVUS configuration 600.

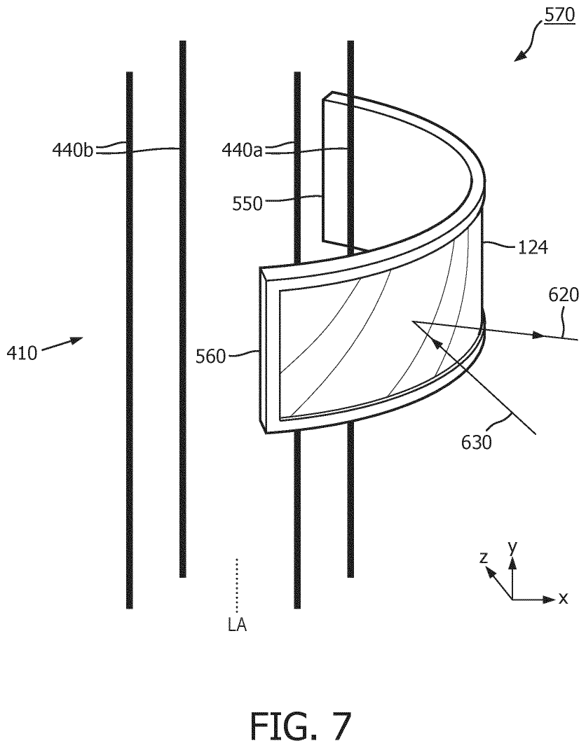

FIG. 7 is a diagrammatic, perspective side view of at least a portion of an example imaging array 124 and expandable support member 410 in the expanded or ICE configuration 570, according to aspects of the present disclosure. In the example shown in FIG. 7, the expandable support member 410 includes support arms 440a that are in contact with (e.g., attached to) the imaging array 124 (or to the flexible substrate, acoustic backing layer, or other layers associated with the imaging array 124). However, because the expandable support member 410 is in an expanded state 570 rather than a compressed state 600, the imaging array 124 is also in an expanded state, such that the first edge 550 and second edge 560 are no longer in contact or proximate to each other. In some embodiments, the imaging array 124 is not elastic and therefore cannot stretch or expand along with the expandable support member. Thus, in contrast with the configuration shown in FIG. 6, some arms 440b of the support member 410 are no longer in contact with the imaging array 124.

In the example shown in FIG. 7, as in the example of FIG. 6, the imaging array 124 still emits ultrasound radiation 620 in a radially outward direction, and receives ultrasound echoes 630 in a radially inward direction. However, the effective radius or bend radius of the imaging array 124 is now larger than in FIG. 6, in proportion with the expansion of the expandable support member 410. It is noted that in the ICE configuration, the imaging array 124 may be too large to traverse some body lumens, but may be of appropriate size to traverse chambers of the heart, or interior volumes of other organs of the body.

Although the struts or arms 440a and 440b are shown as separate structures, some or all arms 440a and 440b may be coupled together at one or more locations (e.g., at the proximal and distal ends of the expendable support member 410). In some embodiments, expansion of the expandable support structure 410 occurs when only a portion of the arms are attached to the transducer array 124 (while other arms remain unattached to the transducer array 124). In other embodiments, depending on the implementation, expansion may be effectuated when all arms are attached to the transducer array 124.

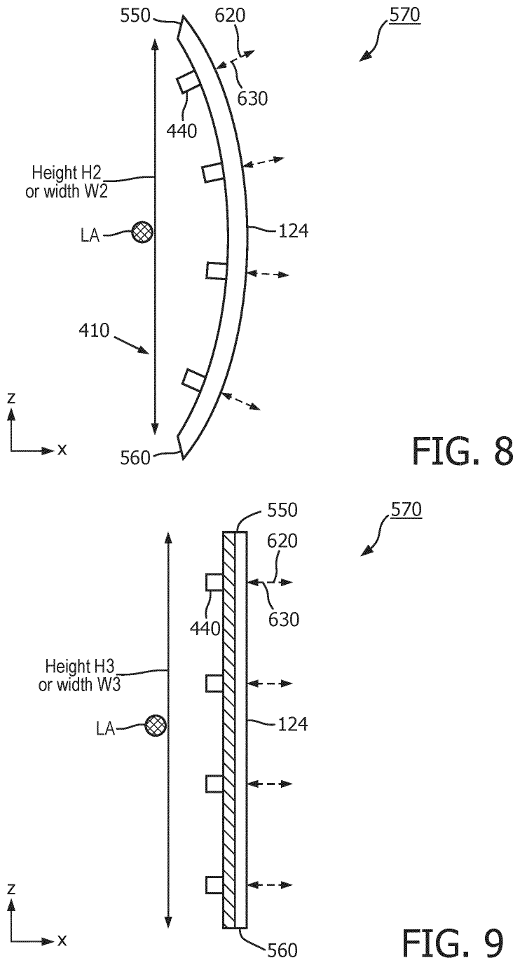

FIG. 8 is a diagrammatic, front cross-sectional view of at least a portion of an example imaging array 124 and expandable support member 410 in the expanded or ICE configuration 570, according to aspects of the present disclosure. Visible are the transducer array 124, the arms 440, the first edge 550, the second edge 560, and the longitudinal axis LA. In the example shown in FIG. 8, unlike FIG. 7, the design of the arms 440 is such that all of the arms 440 remain in contact with the transducer array 124 when the arms 440 are in the expanded state. The first edge 550 thus separates from the second edge 560 until the transducer array forms a convex arc, with a height H2 or width W2 that is greater than the height H1 or width W1 of FIG. 6. In the example shown in FIG. 8, as in the example of FIG. 6, the imaging array still emits ultrasound radiation 620 in a radially outward direction, and receives ultrasound echoes 630 in a radially inward direction. However, the effective radius or bend radius of the imaging array 124 is now larger than in FIG. 6, in proportion with the expansion of the expandable support member 410. Depending on the implementation, there may be additional arms 440 that are not attached to the transducer array 124.

FIG. 9 is a diagrammatic, front cross-sectional view of at least a portion of an example imaging array 124 and expandable support member 410 in the expanded or ICE configuration 570, according to aspects of the present disclosure. Visible are the transducer array 124, the arms 440, the first edge 550, the second edge 560, acoustic backing material 610, and longitudinal axis LA. In the example shown in FIG. 9, like FIG. 8, the design of the expandable support member 410 is such that all of the arms 440 remain in contact with the transducer array 124 when the arms 440 are in the expanded state. The first edge 550 thus separates from the second edge 560 until the transducer array forms a flat planar shape, with a height H3 or width W3 that is greater than the height H1 or width W1 of FIG. 6. In the example shown in FIG. 9, the imaging array emits ultrasound radiation 620 in a lateral direction orthogonal to and away from the imaging array 124, and receives ultrasound echoes 630 in a lateral direction orthogonal to and toward the imaging array 124. In this particular ICE configuration 570, the effective radius or bend radius of the imaging array 124 is very large, or infinite. It is understood that the ICE configuration 570 can assume a continuum of arcuate shapes in between the cylinder of FIG. 6 and the flat plane of FIG. 9. Depending on the implementation, there may be additional arms 440 that are not attached to the transducer array 124

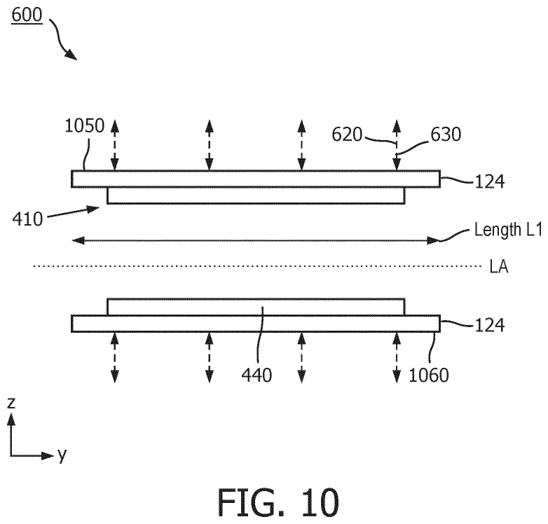

FIG. 10 is a diagrammatic, side cross-sectional view of an example imaging array 124 in the compressed, unexpanded, or IVUS configuration 600, according to aspects of the present disclosure. The imaging array 124 is supported by the arms 440 of the expandable support member 410, and is attached to at least some of the arms 440 by attachment points 450. In the IVUS configuration 600, the imaging array 124 sends ultrasound energy 620 in a radially outward direction, and receives ultrasound echoes 630 in a radially inward direction. In the compressed, unexpanded, or IVUS configuration 600, the imaging array 124 is rolled into a cylindrical shape that is at least approximately concentric with the longitudinal axis LA. The resulting cylindrical shape has a length L1, a proximal end 1050, and a distal end 1060.

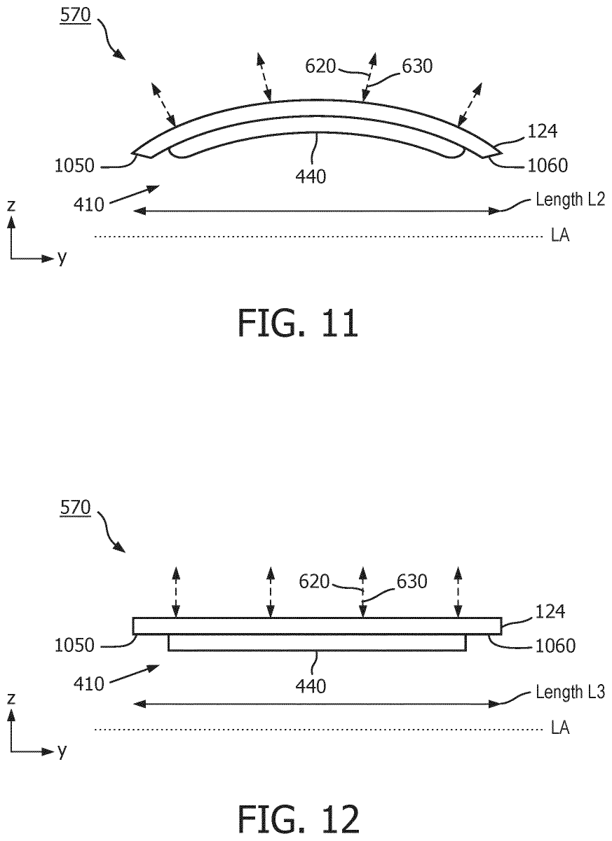

FIG. 11 is a diagrammatic, side cross-sectional view of at least a portion of an example imaging array 124 and expandable support member 410 in an expanded or ICE configuration 570, according to aspects of the present disclosure. Visible are the transducer array 124, the arms 440 of the expandable support member 410, the proximal end 1050, the distal end 1060, and the longitudinal axis LA. In the example shown in FIG. 11, the design of the expandable support member 410 is such that all of the arms 440 remain in contact with the transducer array 124 when the arms 440 are in the expanded state 570. In the non-limiting example of FIG. 11, the transducer array 124 forms an arcuate shape with an axis of curvature perpendicular to the longitudinal axis LA, and a length L2 that is smaller than the length L1 shown in FIG. 10. It is understood that although the curvature shown in FIG. 11 is orthogonal to the curvature shown in FIG. 8, the transducer array 124 may, depending on the implementation, be curved in either of these directions, or in both simultaneously, or along other axes in between those shown in FIGS. 8 and 11. The imaging array emits ultrasound radiation 620 in an outward direction orthogonal to the imaging array 124, and receives ultrasound echoes 630 in an inward direction orthogonal the imaging array 124, as shown.

FIG. 12 is a diagrammatic, side cross-sectional view of at least a portion of an example imaging array 124 and expandable support member 410 in an expanded or ICE configuration 570, according to aspects of the present disclosure. Visible are the transducer array 124, the arms 440 of the expandable support member 410, the proximal end 1050, the distal end 1060, and the longitudinal axis LA. In the example shown in FIG. 12, the design of the expandable support member 410 is such that all of the arms 440 remain in contact with the transducer array 124 when the arms 440 are in the expanded state or ICE configuration 570. In the non-limiting example of FIG. 12, the transducer array 124 forms flat, planar shape with a length L3 that is equal to the length L1 shown in FIG. 10. The imaging array emits ultrasound radiation 620 in an outward direction orthogonal to the imaging array 124, and receives ultrasound echoes 630 in an inward direction orthogonal the imaging array 124, as shown.

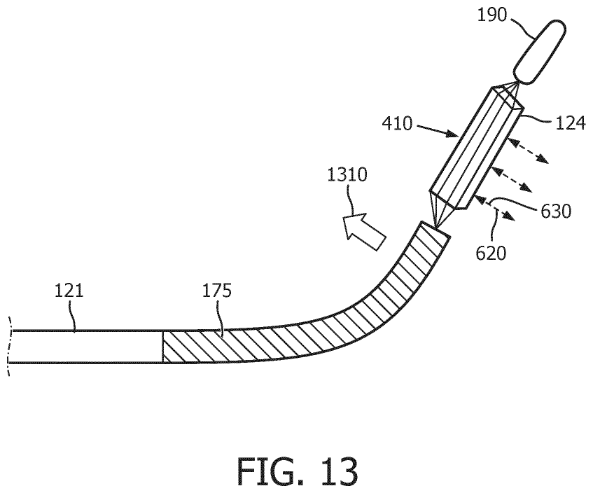

FIG. 13 is a side perspective view of at least a portion of an example hybrid intraluminal imaging device 102 in an expanded or ICE configuration 570, according to aspects of the present disclosure. Visible are the flexible elongate member, articulation section 175, expandable support member 410, transducer array 124, distal tip 190, emitted ultrasound energy 620, and received ultrasound echoes 630. When actuated by a clinician or other user, the articulation section 175 can transition between a straight configuration (as shown for example in FIG. 1) and a bent configuration as shown here in FIG. 13, with arrow 1310 indicating a direction of bending. As described above, in some embodiments, the articulation section 175 is configured to be actuated on more than one axis. When unfurled in ICE mode as shown, the transducer array 124 can for example be used to look at heart valves enface, as well as looking for regurgitation or leaks using doppler ultrasound.

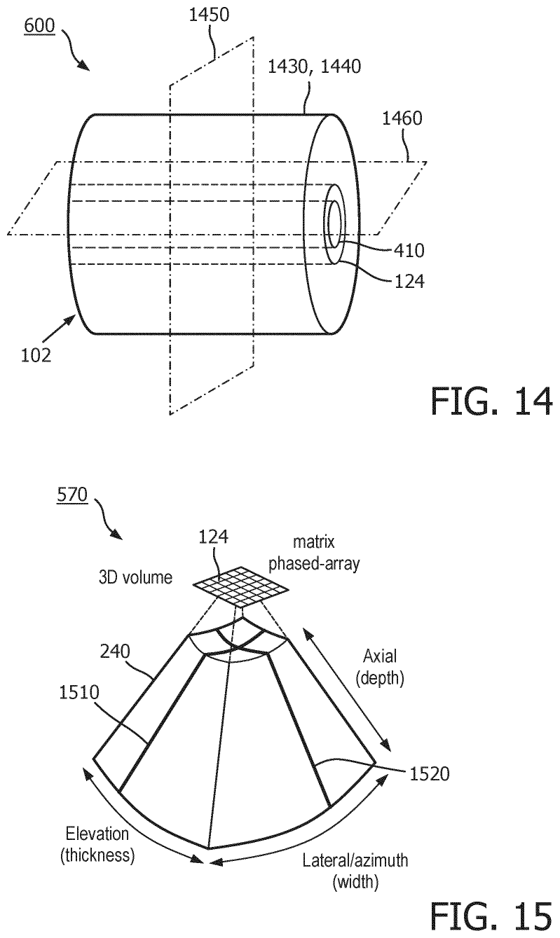

FIG. 14 is a diagrammatic, side perspective view of the viewing region 1430 of an example hybrid intraluminal imaging device 102 in the compressed, unexpanded, cylindrical, or IVUS configuration 600, according to aspects of the present disclosure. Visible are the expandable support member 410 and imaging array 124. The flat, disc-shaped viewing region 230 of FIG. 2A results from a linear (1D) array of transducers. However, in the example of FIG. 14, the hybrid intraluminal imaging device 102 includes a 2D imaging array 124 with multiple rows of transducers (see FIG. 5 for a non-limiting example). Thus, although the viewing region 1430 could be a flat disc (resulting in a 2D cross-sectional image, as shown for example in FIG. 3A) if only one row of transducers were employed, it can also be a cylindrical volume as shown in FIG. 14, if multiple rows of transducers are used in the IVUS mode 600. This can result in a cylindrical 3D image 1440, from which planar cross-sectional images may be taken. Cross section 1450 is a tomographic, lateral, or radial cross section, whereas cross section 1460 is a longitudinal cross section. In IVUS imaging procedures, a longitudinal image of a region of interest can be assembled from a plurality of tomographic images captured at different positions within a body lumen. However, the 2D imaging array 124 of the present disclosure allows a longitudinal image 1460, of at least a portion of a region of interest, to be created without the need to move the imaging device along the body lumen. Other cross sections, at any desired angle, may be taken, depending on the needs of the clinician.

FIG. 15 is a diagrammatic, side perspective view of the viewing region 240 of an example hybrid intraluminal imaging device 102 in the expanded or ICE configuration 600, according to aspects of the present disclosure. The 2D imaging array 124 captures a 3D (e.g., come-shaped or pyramidal) volume 240, which can generate a 3D image, or can be cross-sectioned to produce planar images 1510 or 1520, or other cross sections depending on the needs of the clinician.

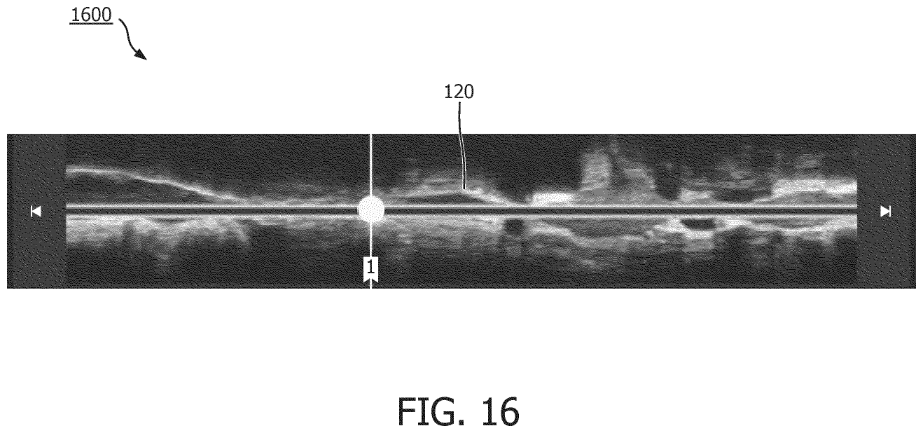

FIG. 16 is a longitudinal cross-sectional view 1600 of at least a portion of an example body lumen 120, according to aspects of the present disclosure. As discussed above, a longitudinal view of a region of interest may be assembled from multiple tomographic IVUS images captured at different positions within the body lumen (e.g., during a pullback procedure), or may be captured as a single image/dataset by a 2D imaging array in a cylindrical or IVUS configuration.

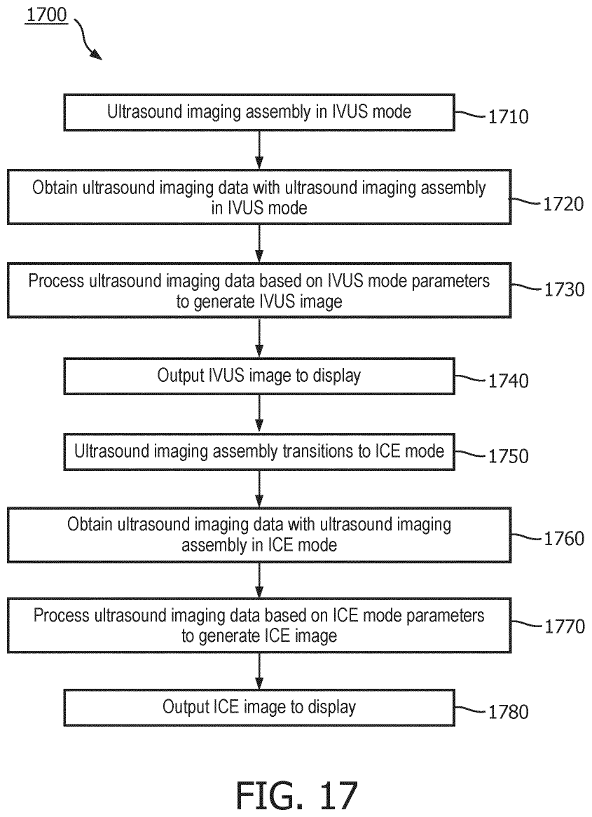

FIG. 17 is a flow diagram of an example hybrid ultrasound imaging method 1700, according to aspects of the present disclosure. It is understood that the steps of method 1700 may be performed in a different order than shown in FIG. 17, additional steps can be provided before, during, and after the steps, and/or some of the steps described can be replaced or eliminated in other embodiments. One or more of steps of the method 1700 can be carried by one or more devices and/or systems described herein, such as components of the system 100, computer 106, and/or processor circuit 1850.

In step 1710, the method 1700 begins with the hybrid ultrasound imaging assembly in the IVUS mode. This may for example be a default state of the device, such that the device can be inserted into a body lumen (e.g., a vein or artery) of a patient. Such insertion may for example be performed by a clinician, who advances the imaging catheter until the imaging assembly reaches a region of interest within the body lumen.

In step 1720, the method 1700 includes obtaining ultrasound imaging data from the hybrid ultrasound imaging assembly in the IVUS mode. This may for example include data representing a series of tomographic images or 3D images captured while the imaging assembly is moved through the body lumen (e.g., during a pullback procedure), or a single 3D image of the region of interest, or otherwise.

In step 1730, the method 1700 includes processing the intraluminal ultrasound data obtained in step 1720, to generate one or more IVUS images, which may be 2D images, 3D images, or 2D cross sections of 3D images. In some cases, IVUS images may include images of the interior of the heart. In an example, this processing may involve scan conversion of 1D or 2D sensor data into a tomographic or circumferential image.

In step 1740, the method 1700 includes outputting the one or more IVUS images to a display, which may for example be viewable by the clinician.

In step 1750, the method 1700 includes transitioning the imaging assembly from the IVUS configuration to the ICE configuration. In some cases, this transitioning is controlled by the user (e.g., a clinician manipulating an actuator such as a lever or dial) and is recognized by hardware, software, and/or firmware elements of the system. In other cases, the transition is requested by the user but is controlled by hardware, software, and/or firmware elements of the system (e.g., via a software-controlled actuator).

In step 1760, the method 1700 includes obtaining ultrasound imaging data from the hybrid ultrasound imaging assembly in the ICE mode. This may for example include data representing one or more 3D images captured while the imaging is positioned within a chamber of the heart, or an interior space of another organ of the body.

In step 1770, the method 1700 includes processing the intraluminal ultrasound data obtained in step 1760, to generate one or more ICE images, which may be 3D images, or 2D cross sections of 3D images. In an example, this processing may involve scan conversion of 2D sensor data into a sector image format.

In step 1780, the method 1700 includes outputting the one or more ICE images to a display, which may for example be viewable by the clinician.

It is understood that instead of transitioning from IVUS mode to ICE mode, the method 1700, or a similar method, may include similar steps to transition from ICE mode to IVUS mode.

Flow diagrams and block diagrams are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular data flow. It is understood that some embodiments of the systems disclosed herein may include additional components, that some components shown may be absent from some embodiments, and that the arrangement of components may be different than shown, resulting in different data flows while still performing the methods described herein.

Similarly, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be necessary in order to execute the method in real time or near-real time as described herein.

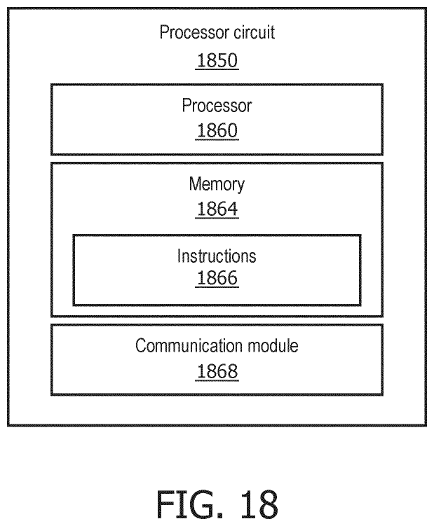

FIG. 18 is a schematic diagram of a processor circuit 1850, according to aspects of the present disclosure. The processor circuit 1850 may be implemented in the ultrasound imaging system 100, or other devices or workstations (e.g., third-party workstations, network routers, etc.), or on a cloud processor or other remote processing unit, as necessary to implement the method. As shown, the processor circuit 1850 may include a processor 1860, a memory 1864, and a communication module 1868. These elements may be in direct or indirect communication with each other, for example via one or more buses.

The processor 1860 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 1860 may also comprise another hardware device, a firmware device, or any combination thereof configured to perform the operations described herein. The processor 1860 may also be implemented as a combination of computing devices, e.g., a combination of a DSP and a microprocessor, a plurality of microprocessors, one or more microprocessors in conjunction with a DSP core, or any other such configuration.

The memory 1864 may include a cache memory (e.g., a cache memory of the processor 1860), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1864 includes a non-transitory computer-readable medium. The memory 1864 may store instructions 1866. The instructions 1866 may include instructions that, when executed by the processor 1860, cause the processor 1860 to perform the operations described herein. Instructions 1866 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1868 can include any electronic circuitry and/or logic circuitry to facilitate direct or indirect communication of data between the processor circuit 1850, and other processors or devices. In that regard, the communication module 1868 can be an input/output (I/O) device. In some instances, the communication module 1868 facilitates direct or indirect communication between various elements of the processor circuit 1850 and/or the ultrasound imaging system 100. The communication module 1868 may communicate within the processor circuit 1850 through numerous methods or protocols. Serial communication protocols may include but are not limited to US SPI, I²C, RS-232, RS-485, CAN, Ethernet, ARINC 429,MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or other appropriate subsystem.

External communication (including but not limited to software updates, firmware updates, preset sharing between the processor and central server, or readings from the ultrasound device) may be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

A number of variations are possible on the examples and embodiments described above. For example, rather than comprising arms made of a shape memory alloy, the expandable support member could be or could comprise any other type of expanding structure, including without limitation: a balloon, a spring, a bladder, a folded or spiraled structure, a compressible foam, and/or a mechanically articulated expanding structure, along with appropriate actuators and control mechanisms as would occur to a person of ordinary skill in the art.

The technology described herein may be used for intravascular and/or intracardiac procedures.

The logical elements making up the embodiments of the technology described herein are referred to variously as operations, steps, objects, elements, components, modules, portions, or otherwise. Furthermore, it should be understood that these may occur or be performed in any order or arrangement, unless explicitly claimed otherwise or a specific order or arrangement is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the hybrid transducer assembly. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the hybrid transducer as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

1. An intraluminal imaging device, comprising:
a flexible elongate member configured to be positioned within a patient; and
an ultrasound imaging assembly coupled to a distal portion of the flexible elongate member and comprising:
an expandable support member comprising a first state and a second state; and
a transducer array coupled to the expandable support member,
wherein, in the first state of the expandable support member, the transducer array comprises a first shape, and in the second state of the expandable support member, the transducer array comprises a second shape.

2. The device of claim 1, wherein the expandable support member comprises a shape memory alloy.

3. The device of claim 1,
wherein the flexible elongate member comprises a tension wire, and
wherein changing a tension of the tension wire causes the expandable support member to change from the first state to the second state.

4. The device of claim 1, wherein the transducer array comprises a first edge and an opposite second edge.

5. The device of claim 4, wherein the first shape is substantially cylindrical such that the first edge is in contact with the second edge or proximate to and facing substantially toward the second edge.

6. The device of claim 4, wherein the second shape is a planar or open arcuate shape such that the first edge is spaced from, and not facing toward, the second edge.

7. The device of claim 1, wherein the transducer array comprises a plurality of capacitive micromachined ultrasonic transducers (CMUTs).

8. The device of claim 7, wherein the first shape of the transducer array comprises an intravascular ultrasound (IVUS) configuration.

9. The device of claim 7 wherein the second shape of the transducer array comprises an intracardiac echography (ICE) configuration.

10. The device of claim 1, wherein the transducer array is a two-dimensional (2D) transducer array.

11. The device of claim 1,
wherein the imaging assembly further comprises a flexible substrate coupled to the expandable support member, and
wherein the transducer array coupled to the flexible substrate.

12. The device of claim 1, wherein the flexible elongate member further comprises a pullwire coupled to and configured to deflect the distal portion of the flexible elongate member.

13. The device of claim 1, wherein a diameter of the transducer array is larger in the second shape of the transducer array than in the first shape of the transducer array.

14. The device of claim 1, wherein the expandable support member comprises a first plurality of arms that are attached to the transducer array and a second plurality of arms that are not attached to the transducer array.

15. A method, comprising:
providing an intraluminal imaging device comprising:
a flexible elongate member configured to be positioned within a first body lumen and a second body lumen of a patient; and
an ultrasound imaging assembly coupled to a distal portion of the flexible elongate member and comprising:
an expandable support member in a first state; and
a transducer array coupled to the expandable support member such that, in the first state of the expandable support member, the transducer array comprises a first shape for imaging while positioned within the first body lumen; and
transitioning the expandable support member to a second state such that the transducer array comprises a second shape for imaging while positioned within the second body lumen.

16. The device of claim 1, further comprising:
a tension wire configured to change the expandable support member between & the first state of expansion and a the second state of expansion.

17. The device of claim 1, wherein:
the flexible elongate member is configured to be positioned within a blood vessel of the patient and a heart chamber of the patient, and
in the first state of the expandable support member, the transducer array is in a substantially cylindrical shape suitable for intravascular ultrasound (IVUS) imaging within the blood vessel, and in the second state of expansion of the expandable support member, the transducer array is in a substantially planar or hemicylindrical shape suitable for intracardiac echography (ICE) imaging within the heart chamber.

18. The device of claim 1, further comprising:
an actuating mechanism configured to change the expandable support member between the first state and a second state, and wherein the actuating mechanism comprises a tension wire or a pullwire.

19. The method of claim 15, wherein the intraluminal imaging device further comprises an actuating mechanism that comprises a tension wire or a pullwire, and the method further comprises actuating the actuating mechanism to transition the expandable support member to the second state.

20. The method of claim 15, wherein:
the first body lumen is a blood vessel and the second body lumen is a heart chamber, and
in the first state of the expandable support member, the transducer array is in a substantially cylindrical shape suitable for intravascular ultrasound (IVUS) imaging within the blood vessel, and in the second state of expansion of the expandable support member, the transducer array is in a substantially planar or hemicylindrical shape suitable for intracardiac echography (ICE) imaging within the heart chamber.
